(19) United States
(12) Patent Application Publication
(10) Pub. No.: US 2025/0283175 A1
(43) Pub. Date: Sep. 11, 2025

(54) METHODS FOR EVALUATION OF EARLY STAGE ORAL SQUAMOUS CELL CARCINOMA

(71) Applicant: LOMA LINDA UNIVERSITY, Loma Linda, CA (US)

(72) Inventors: Chi T. Viet, Loma Linda, CA (US); Bradley E. Aouizerat, Loma Linda, CA (US)

(73) Assignee: LOMA LINDA UNIVERSITY, Loma Linda, CA (US)

(21) Appl. No.: 18/261,537

(22) PCT Filed: Jan. 14, 2022
(86) PCT No.: PCT/US22/70208

§ 371 Date: Jul. 14, 2023

(51) Int. Cl. C12Q 1/6886

(52) U.S. Cl. CPC C12Q 1/6886; C12Q 2600/118; C12Q 2600/154

(57) ABSTRACT

Provided here are methods of prognosis of oral squamous cell carcinoma in an individual, methods of providing decision support for suitable treatment regimens, and methods of monitoring responsiveness to treatment. These methods include the step of determining a high-risk epigenetic and clinicopathologic score for oral cancer from a sample from the individual. The sample can be a brush biopsy sample.

### CROSS-REFERENCE TO RELATED APPLICATIONS

This application claims the benefit of and priority to U.S. Provisional Application No. 63/199,655, filed on Jan. 14, 2021, which is incorporated herein by reference in its entirety.

### TECHNICAL FIELD

This disclosure relates to systems and methods for evaluation, diagnostics, prognostics, and treatment support for oral squamous cell carcinoma (OSCC).

### BACKGROUND

Each year 30,000 patients are diagnosed with oral cavity squamous cell carcinoma (OSCC), and unfortunately the incidence is on the rise. Even for these early stage patients, the five-year survival rate is 60%. Poor survival rates are in part due to inaccurate risk prediction. Early stage OSCC is primarily treated with surgical resection of the cancer, with or without adjuvant treatments, such as an elective lymphadenectomy, radiation, or chemoradiation, for patients with high risk features. Currently, risk prediction to assign adjuvant treatment is entirely based on clinicopathologic information. Multiple retrospective and prospective studies have shown that these standard clinicopathologic factors have moderate accuracy with a concordance statistic (c-statistic) of 0.7. Genome-wide association studies to date have not produced a viable biomarker. Shortcomings of these studies include a failure to use a clinically translatable array platform, and a failure to quantify methylation in real time, as cancer treatment is occurring. There is a pressing need to develop more precise risk assessment methods to appropriately tailor clinical treatment.

### SUMMARY

Provided here are diagnostic and therapeutic methods for the treatment of OSCC. For example, provided are methods of prognosis of OSCC and determination of suitable treatment regimens and methods of monitoring responsiveness to treatment. In an embodiment, the method of prognosis for an individual having OSCC includes the step of determining a high-Risk Epigenetic And clinicopathologic Score for Oral cancer (REASON) score from a biological sample from the individual. Methods also include a noninvasive approach to collect a biological sample from a subject for evaluation of OSCC in the subject. An embodiment also includes a method of collection of a sample from the patient for evaluation of the disease and determining prognosis for the patient. In an embodiment, the biological sample can be a collection of cells from the suspected cancerous tissue, or from saliva or blood or other bodily fluid from the individual. In an embodiment, the biological sample is obtained by a brush biopsy. In an embodiment, the sample is a brush swab sample. In an embodiment, the subject is diagnosed to have early-stage (I/II) OSCC based on evaluation of the methylome of the biological sample. The REASON score is a combination of a plurality of non-molecular variables and a plurality of methylation patterns of a plurality of genes. The plurality of non-molecular variables include age, sex, race, tobacco use, alcohol use, histologic grade, stage, perineural invasion (PNI), lymphovascular invasion (LVI), and margin status. The plurality of genes whose methylation patterns are determinative of the REASON score include two or more of ABCA2 (ATP-binding cassette sub-family A member 2), CACNA1H (Calcium Voltage-Gated Channel Subunit Alphal H), CCNJL (Cyclin-J-Like), GPR133 (Adhesion G-Protein-Coupled Receptor 133), HGFAC (hepatocyte growth factor activator), HORMAD2 (HORMA domain containing protein 2), MCPHI (Microcephalin 1), MYLK (Myosin Light Chain Kinase), RNF216 (Ring finger protein 216), SOX8 (SRY-box transcription factor 8), TRPA1 (Transient Receptor Potential Cation Channel Subfamily A Member 1), and WDR86 (WD Repeat Domain 86).

Embodiments include a method of providing a treatment regimen recommendation based on prognosis of OSCC. The method includes the step of determining a REASON score from a sample from the individual, wherein the REASON score from the sample that is at or above a reference REASON score indicates a poor prognosis. The REASON score for the clinicopathologic component ranges from zero to nine (for the nine dichotomized risk factors-race, sex, seven risk factors [PNI, tumor grade, margin status, LVI, stage, current tobacco smoking, history of alcohol use]) and zero to twenty-six for the 13 CpG epigenetic sites (categorized as tertiles). The total REASON score ranges from zero to thirty-five, by combining the clinicopathologic score with the epigenetic score. In an embodiment, the reference REASON score is a median cutoff range of the total REASON score as used to categorize participants into low risk and high risk subgroups. In an embodiment, the reference REASON score of 17 is used to categorize participants into low risk and high risk subgroups.

Embodiments include a method for identifying an individual having an early-stage (I/II) OSCC who may benefit from a surgical treatment by determining a REASON score from a sample from the individual. The REASON score provides a decision support tool for a healthcare professional and a patient to evaluate and select treatment regimens, such as one or more of an elective neck dissection, radiation, or chemotherapy. Embodiments include a method for selecting a therapy for an individual having OSCC. In an embodiment, the method includes determining a REASON score from a sample from the individual. The REASON score from the sample being at or above a reference REASON score indicates the individual as one who may benefit from one or more treatment options, such as one or more of a neck dissection, radiation, or chemotherapy. In an embodiment, the reference REASON score is a median cutoff range of the total REASON score as used to categorize participants into low risk and high risk subgroups. In an embodiment, the reference REASON score of 17 is used to categorize participants into low risk and high risk subgroups.

Embodiments include methods of risk stratification of an individual having oral squamous cell carcinoma (OSCC) using the REASON score. One such method includes the step of determining a high-Risk Epigenetic And clinicopathologic Score for Oral cancer (REASON) score from a biological sample from the individual; and classifying the individual as having a high risk of OSCC-related mortality in response to the REASON score for the individual with OSCC being above a reference REASON score from a healthy individual.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

Embodiments will be readily understood by the following detailed description in conjunction with the accompanying drawings. Embodiments are illustrated by way of example and not by way of limitation of the accompanying figures.

FIG. 1 is a flowchart of a method of analysis of the methylation array data from the TCGA cohort, according to an embodiment.

FIG. 2 is a heat map and hierarchical clustering of differentially methylated genes demonstrates distinct methylation signature in high-risk vs. low-risk OSCC patients. It is a heat map of the 12 top differentially methylated genes between patients who survived to five years vs. those who died in The Cancer Genome Atlas (TCGA) cohort.

FIGS. 3A and 3B are representations from a functional network analysis mapping. Functional enrichment analysis identifies the aggregation of differentially methylated genes on to three pathways. FIG. 3A is a dot plot of differentially enriched genes that map to the top ten most differentially perturbed methylated pathways (p_{adjusted}<0.05). FIG. 3B is a representation of the top three most statistically differentially methylated pathways as identified by a circle in grey and the fold change in differential methylation of component genes is rendered in color ranging from negative (green) to positive (red) fold change for each gene. The size of each circle is based on the number of genes.

FIG. 4A is a graphical representation of the coverage in all CpGs that demonstrates an inflection point at 10× coverage. FIG. 4B is a graphical representation of the number of quantified CpGs in both swab and tissue samples of cancer and normal subjects. Using 10× read depth as a cutoff, the number of quantified CpG sites was determined in each sample. FIG. 4C is a graphical representation of the average mapping efficiency for brush swabs and for tissues. The average mapping efficiency was 89.45% for brush swabs and 90% for tissues, with no significant difference between the two sampling methods. FIG. 4D is a set of pie chart representations of the relative genic locations of the CpGs profiled by MC-Seq (left) and CpGs covered by the EPIC array that were profiled (right). MC-Seq provided more robust coverage of functional gene regions than the EPIC array.

FIGS. 5A and 5B are scatterplots demonstrating the correlation between tissue and brush swab biopsies for cancer and normal sites, respectively, of the 3 patients. The correlation values are noted. FIG. 5C is a graphical representation of the methylation difference between cancer and normal samples quantified with MC-Seq, visualized using box plots (median, quartiles, maximum and minimum whiskers).

FIGS. 6A-6L are representative M-bias coverage plots demonstrating that the characteristic M-value bias is consistent in cancer samples as compared to normal samples as well as samples obtained from a brush swab as compared to a tissue biopsy.

### DETAILED DESCRIPTION

Almost all of the cancers in the oral cavity and oropharynx are squamous cell carcinomas (OSCC) that start in squamous cells, which form the lining of the mouth and throat. Oral cancer is on the rise, increasing by two thirds in the past 20 years. Each year 30,000 Americans are diagnosed with oral cavity squamous cell carcinoma and 80% of newly diagnosed cases are early stage I/II without regional lymph node involvement or distant metastasis. Even for early stage oral cancer patients, the five-year survival rate is as low as 60%. OSCC patients are treated with surgical resection of the cancer and neck lymphadenectomy, followed by adjuvant radiation with or without chemotherapy and immunotherapy based on risk stratification. However, with the current clinical practices of relying solely on clinicopathologic information, risk prediction and survival, remain poor. Up to 40% of OSCC patients, even those who present with early-stage cancer, die within five years. This poor survival rate is in contrast to other cancers, or even other head and neck cancer subtypes, such as oropharyngeal SCC. There is a need to develop robust prognostic methods that combine both clinicopathologic data with molecular signatures to stratify OSCC patients into high and low risk categories, and will provide clinical decision support about adjuvant chemotherapy and radiation, and ultimately improve survival.

Embodiments include methods of sample collection to quantify OSCC-specific methylation features. One such method includes a brush swab biopsy to serve as a robust noninvasive method to quantify cancer-specific methylation features. In certain embodiments, the method includes subsequent processing of the sample from the brush swab biopsy through a Methyl-Capture Sequencing (MC Seq) process to establish a methylation signature. This signature is evaluated in combination with clinicopathologic factors to arrive at a REASON score, which is used to determine a risk of mortality and provide decision support for an appropriate treatment regimen.

Disclosed here are methods wherein gene methylation signatures are combined with clinicopathologic factors to form a composite molecular and non-molecular signature with high prognostic performance in determining risk of 5-year mortality in early stage (I/II) OSCC patients. Clinicopathologic data were analyzed from an internal retrospective cohort of 515 OSCC patients as well as a cohort of 58 patients from TCGA. The top clinicopathologic factors that were highly predictive of 5-year mortality in these two cohorts were determined. Available methylation array data in the TCGA cohort were analyzed and twelve genes were identified that were differentially methylated between the OSCC patients who died by 5 years and those who survived. The relevant clinicopathologic factors with the twelve-gene methylation signature were combined into a risk score—the REASON score. Its predictive performance was evaluated to identify early-stage OSCC patients who died within five years of diagnosis.

Embodiments include a method of providing a treatment regimen recommendation based on prognosis of OSCC. The method includes the step of determining a REASON score from a sample from the individual, wherein the REASON score from the sample that is at or above a reference REASON score indicates a poor prognosis. The REASON score for the clinicopathologic component ranges from 0-9 (for the 9 dichotomized risk factors—race, sex, 7 risk factors [PNI, tumor grade, margin status, LVI, stage, current tobacco smoking, history of alcohol use]) and for the 13 CpG epigenetic sites 0-26 (categorized as tertiles). The total REASON score range is 0-35, by combining the clinicopathologic score with the epigenetic score. In an embodiment, the reference REASON score is a median cutoff range of the total REASON score as used to categorize participants into low risk and high risk subgroups. In an embodiment, the reference REASON score of 17 is used to categorize participants into low risk and high risk subgroups. The method can further include the step of proposing a treatment for the subject based on the REASON score, wherein the treatment is one or more of at least a partial neck resection, an active therapy selected from radiation treatment, chemotherapy, immunotherapy, and a combination thereof; and active surveillance. In certain embodiments, the REASON score can be used for monitoring the patient's responsiveness to a selected treatment regimen.

Embodiments include a method for identifying an individual having an early-stage (I/II) OSCC who may benefit from a surgical treatment by determining a REASON score from a sample from the individual. The REASON score provides a decision support tool for a healthcare professional and a patient to evaluate and select treatment regimens, such as an elective neck dissection, radiation, immunotherapy, or chemotherapy. Embodiments include a method for selecting a therapy for an individual having OSCC. In an embodiment, the method includes determining a REASON score from a sample from the individual. The REASON score from the sample being at or above a reference REASON score indicates the individual as one who may benefit from one or more treatment options, such as neck dissection, radiation, immunotherapy, or chemotherapy. In certain embodiments, the score will be determined empirically based on survival status. In an embodiment, the reference REASON score is a median cutoff range of the total REASON score as used to categorize participants into low risk and high risk subgroups. In an embodiment, the reference REASON score of 17 is used to categorize participants into low risk and high risk subgroups.

Embodiments also include an evaluation kit that includes at least two or more primers and/or probes for determining the methylation pattern of two or more of ABCA2, CACNA1H, CCNJL, GPR133, HGFAC, HORMAD2, MCPH1, MYLK, RNF216, SOX8, TRPA1, and WDR86. This evaluation kit can also contain the instructions for determining the methylation pattern of two or more of ABCA2, CACNA1H, CCNJL, GPR133, HGFAC, HORMAD2, MCPH1, MYLK, RNF216, SOX8, TRPA1, and WDR86. This evaluation kit can also contain the instructions for determining a CpG epigenetic score. This evaluation kit can also contain the instructions for determining a REASON score based on the CpG epigenetic score and plurality of non-molecular variables includes one or more of age of the individual, sex of the individual, race of the individual, tobacco use by the individual, alcohol use by the individual, histologic grade of the OSCC, stage of the OSCC, perineural invasion (PNI), lymphovascular invasion (LVI), and margin status of the OSCC. Embodiments also include methods of use of these evaluation kits for risk stratification of a patient with OSCC.

As used herein, “treating” or “treatment” means complete cure or incomplete cure, or it means that the symptoms of the underlying disease or associated conditions are at least affected, prevented, reduced, eliminated and/or delayed, and/or that one or more of the underlying cellular, physiological, or biochemical causes or mechanisms causing the symptoms are affected, prevented, reduced, delayed and/or eliminated. It is understood that reduced or delayed, as used in this context, means relative to the state of the untreated disease, including the molecular state of the untreated disease, not just the physiological state of the untreated disease. In certain embodiment, determination of the REASON score is part of a comprehensive risk stratification strategy for treating a subject.

Embodiments include methods for risk stratification of a OSCC subject using brush swab samples and MC-Seq to noninvasively determine the methylation signature of an OSCC patient at the time of diagnosis. The methods include the steps of collecting a biological sample using a brush swab, determining a REASON score from the biological sample, which is a combination of a plurality of non-molecular variables and a plurality of methylation patterns of a plurality of genes, and providing a risk stratification in response to the REASON score. The plurality of non-molecular variables include age, sex, race, tobacco use, alcohol use, histologic grade, stage, perineural invasion (PNI), lymphovascular invasion (LVI), and margin status. The plurality of genes whose methylation patterns are determinative of the REASON score include two or more of ABCA2, CACNA1H, CCNJL, GPR133, HGFAC, HORMAD2, MCPH1, MYLK, RNF216, SOX8, TRPA1, and WDR86. This improved stratification of the subject results in better supported primary treatment decisions.

Described here is the patient selection and data collection process in support of development of the REASON score. The patients were selected from an existing OSCC database compiled at the institution at which they were treated. Collection of clinical data for this database was approved by the Institutional Review Board at each institution, which included Loma Linda University (LLU), and Columbia University Irving Medical Center (CUIMC), Portland Providence Medical Center (PPMC), University of Illinois Chicago (UIC), and University of Alabama at Birmingham (UAB). The search was limited to only oral cavity sub-sites, including oral tongue, maxillary and mandibular gingiva, hard palate, floor of mouth, buccal mucosa, and lip mucosa. Clinical and pathologic stages were recorded based on the American Joint Committee on Cancer (AJCC) Eighth Edition Staging Manual. All patients had stage I or II (i.e., T1N0M0 or T2N0M0) biopsy-confirmed OSCC. De-identified patient clinicopathologic characteristics were used in the data interpretation. The following information were collected from the chart review: age, sex, race, smoking and alcohol use, TNM classification, tumor location, pathologic characteristics [i.e., perineural invasion (PNI), lymphovascular invasion (LVI), margin status, histologic grade], and treatment modalities received in addition to tumor ablation (i.e., neck lymphadenectomy, radiation therapy with or without chemotherapy). The internal cohort of 515 patients and TCGA cohort of 58 patients consisted of patients with early stage (I or II) OSCC based on their pathologic TNM classification. Table 1 details their demographic and clinicopathologic characteristics. Statistical tests and p-values are indicated. Abbreviations: AJCC=American Joint Committee on Cancer; NOS=not otherwise specified; SD=standard deviation; TCGA=The Cancer Genome Atlas.

TABLE 1
Patient Demographics and Clinicopathologic Characteristics

| | TCGA | | Internal cohort | | Chi-square | |
|---|---|---|---|---|---|---|
| | (n = 58) | | (n = 515) | | test (df) | p-value |
| Tumor location | | | | | | |
| Alveolus, NOS | 3 | (5.17%) | 9 | (1.75%) | 77.51 (8) | <0.001 |
| Buccal mucosa | 3 | (5.17%) | 23 | (4.47%) | | |
| Floor of mouth | 10 | (17.24%) | 65 | (12.65%) | | |
| Hard palate | | | 24 | (4.67%) | | |
| Lip mucosa | 2 | (3.45%) | 21 | (4.09%) | | |
| Mandibular alveolus | | | 53 | (10.31%) | | |
| Maxillary alveolus | | | 26 | (5.06%) | | |
| Oral cavity, NOS | 7 | (12.07%) | | | | |
| Tongue | 33 | (56.90%) | 293 | (57.00%) | | |
| Sex | | | | | | |
| Female | 23 | (39.66%) | 241 | (46.80%) | 1.07 (1) | 0.301 |
| Male | 35 | (60.34%) | 274 | (53.20%) | | |
| Age | | | | | | |
| Mean (SD) | 64.01 | (12.46) | 65.20 | (14.47) | t (df = | 0.55 |
| | | | | | 571) = −0.60 | |
| Race | | | | | | |
| Asian | 4 | (7.14%) | 12 | (2.60%) | Fisher's | 0.138 |
| Black | 0 | | 13 | (2.81%) | exact test | |
| White | 52 | (92.86%) | 428 | (92.64%) | | |
| Other | 0 | | 9 | (1.95%) | | |
| Ethnicity | | | | | | |
| Hispanic | 2 | (3.57%) | 89 | (22.25%) | 10.73 (1) | 0.001 |
| Non-Hispanic | 54 | (96.43%) | 311 | (77.75%) | | |
| Tobacco use | | | | | | |
| Never smoker | 19 | (33.93%) | 186 | (50.13%) | 5.12 (2) | 0.077 |
| Previous smoker | 25 | (44.64%) | 124 | (33.42%) | | |
| Current smoker | 12 | (21.43%) | 61 | (16.44%) | | |
| Alcohol use | | | | | | |
| No | 22 | (38.60%) | 227 | (60.05%) | 9.32 (1) | 0.002 |
| Yes | 35 | (61.40%) | 151 | (39.95%) | | |
| Survival at 5 years | | | | | | |
| Alive | 50 | (86.21%) | 186 | (63.05%) | 11.73 (1) | 0.001 |
| Dead | 8 | (13.79%) | 109 | (36.95%) | | |
| Tumor grade | | | | | | |
| Moderate/poor | 47 | (81.03%) | 297 | (59.64%) | 10.08 (1) | 0.001 |
| Well | 11 | (18.97%) | 201 | (40.36%) | | |
| Margin status | | | | | | |
| Negative | 45 | (78.95%) | 342 | (68.26%) | 2.75 (1) | 0.097 |
| Close (<5 mm)/positive | 12 | (21.05%) | 159 | (31.74%) | | |
| Perineural invasion | | | | | | |
| No | 28 | (65.12%) | 390 | (88.64%) | 18.61 (1) | <0.001 |
| Yes | 15 | (34.88%) | 50 | (11.36%) | | |
| Lymphovascular invasion | | | | | | |
| No | 40 | (93.02%) | 244 | (82.71%) | 2.97 (1) | 0.085 |
| Yes | 3 | (6.98%) | 51 | (17.29%) | | |
| AJCC pathologic stage | | | | | | |
| Stage I | 18 | (31.03%) | 330 | (64.08%) | 23.87 (1) | <0.001 |
| Stage II | 40 | (68.97%) | 185 | (35.92%) | | |

The TCGA cohort was 60% male, 93% white, and had a mean age of 64. The majority of patients (68%) were current or previous smokers and 61% of patients used alcohol. Tumor sub-sites included the oral tongue, alveolar ridge, buccal mucosa, or floor of mouth; 57% of the TCGA cohort consisted of oral tongue SCC, with the remainder distributed amongst other sub-sites. With regard to pathologic staging, 31% were stage I and 69% were stage II. In terms of tumor grade, 19% had well-differentiated tumors, with the remaining 81% had either moderately or poorly differentiated tumors. PNI was present in 35%, LVI was present in 6.9%, and positive or close margins was present in 21% of cases. Five-year survival was 86%. The significant differences between the TCGA cohort and internal cohort are listed in Table 1. Gender, age, self-reported race, and tobacco use were not different between the two cohorts. The internal cohort featured a greater proportion of patients who self-reported Hispanic ethnicity (22% vs 3.6%, p=0.001). The internal cohort had significantly fewer patients who consumed alcohol (40% vs 61%, p=0.002). There were significant differences in tumor location. While the proportion of patients with tongue SCC was the same in both cohorts (57%), the internal cohort had a higher percentage of alveolar (gingival) SCC than the TCGA cohort (17% vs 5%, p<0.001). There were also differences in tumor grade, with a higher of the internal cohort having well-differentiated tumors (40% vs 19%; p=0.001). A lower percentage in the internal cohort had PNI compared to the TCGA cohort (11% vs 35%; p<0.001). Along the same lines, there were also significantly more patients with a lower pathologic stage in the internal cohort (64% vs 31%, p<0.001). However, despite having earlier-stage, more well-differentiated tumors with lower PNI, the risk of death was significantly higher in the internal cohort (37% vs 14%; p=0.001).

The c-index was calculated using different clinicopathologic factors. The clinicopathologic features with the highest predictive ability among the two cohorts were age, race, sex, tobacco use, alcohol use, histologic grade, stage, PNI, LVI, and margin status. This panel of 10 non-molecular features predicted 5-year mortality risk with a c-index=0.72 for the TCGA cohort, c-index=0.66 for the internal cohort. Despite the reported differences in clinicopathologic characteristics between the two groups, there were no significant differences in prognostic performance. The two groups combined had a c-index=0.67 in predicting 5-year mortality. The low c-index is consistent with previous clinical and biomarker studies, which have demonstrated that clinicopathologic factors alone could not sufficiently assess disease risk as defined by a c-index of ≥0.8. Current clinical practices rely solely on these clinicopathologic factors for risk assessment and treatment decisions.

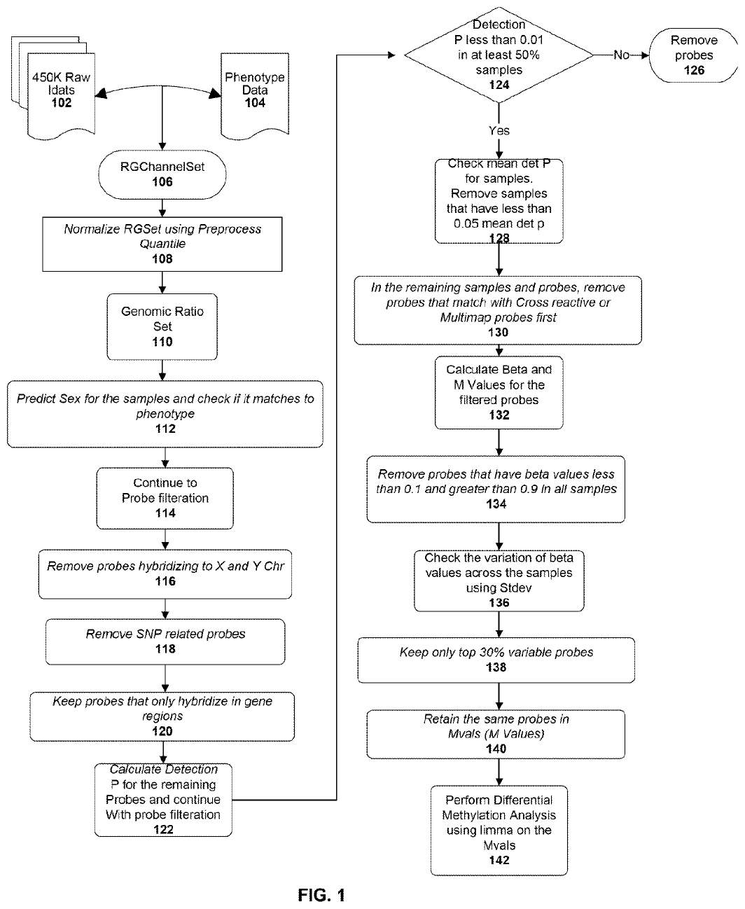

An analysis of methylation data from early-stage OSCC patients in the TCGA database was performed. DNA methylation data pre-processing, quality control filtering, and normalization (inclusive of batch correction and surrogate variable analysis) were conducted employing the minfi package in the R bioconductor package. The minfi package is a flexible and comprehensive bioconductor package for the analysis of Infinium® DNA methylation microarrays. Differential methylation analysis was performed using the limma package in the R package. The Illumina Infinium Methylation 450K Array data analyses is described in FIG. 1. FIG. 1 is a flowchart of a method of analysis of the methylation array data from the TCGA cohort, according to an embodiment.

In a method 100, two datasets—the 450K array 102 and the phenotype data 104 were loaded into the RGChannelSet 106 of the minfi package. These constitute raw (unprocessed) data from a two color micro array; specifically an Illumina methylation array. The RGset data is then normalized 108 using the preprocessQuantile function that implements stratified quantile normalization preprocessing for Illumina methylation microarrays. The data is then processed 110 by a genomic ratio set function where methylation microarrays are mapped to a genomic location. In the next step 112, the sex of the samples is predicted and then checked against the phenotype of the samples. Step 112 is a quality control step to determine that the sample and output are consistent, by identifying samples that are discordant between self-reported and biological sex. Then the data was subjected to a probe filtration step 114. Briefly, out of a total of 485,512 probes, probes that hybridized to the X or Y chromosomes were removed 116, leaving 473,864 probes. An additional 17,351 probes related to single nucleotide polymorphisms (SNPs) were removed 118 and 111,977 probes that did not map to gene regions were removed 120. The p value was calculated for the remaining probes as part of the next step 122 of the probe filtration process. When a detection p value of <0.01 in at least 50% of the samples was determined in step 124, those probes that had a detection p value of more than 0.01 in at least 50% of the samples were removed in step 126. From the remaining 344,536 probes, those probes that had a detection p value of <0.01 in at least 50% of the samples were retained in step 128. The probes that were cross reactive or mapped to multiple genomic positions were then filtered in step 130, leaving 324,465 probes.

The beta and M values for the filtered probes were then calculated in step 132. Beta values are the raw estimates of methylation at each CpG site (range 0-1). An M value is a different estimate of the same methylation state that has better statistical properties for analysis. The probes with a beta value of <0.1 across all samples or >0.9 across all samples were excluded in step 134, leaving 317,016 probes. Using the patient's survival status as the outcome variable, batch correction using surrogate variable analysis was performed. The variation of beta values across the samples was analyzed in step 136. Surrogate variables with a correlation of higher than 0.2 with survival status were excluded (3 of 14 surrogate variables identified). Surrogate variable analysis is employed to identify patterns in the data that are unrelated to the outcome of interest (e.g., batch effects), but cause unwanted variation that could influence the analysis. Surrogate variables are estimated from high-dimensional data and used as covariates to adjust for these unwanted sources of variation. The top 30% most variable methylated probes were then selected in step 138, which resulted in a total number of 95,104 probes spanning 4,544 genes retained for differential methylation analysis. The same probes were retained in Mvalues in step 140. Beta values are used to interpret the methylation state of a CpG site, but M values are used for the statistical analysis of the same site. Differential methylation analysis using the limma feature on the M values was performed using the R bioconductor package, wherein the limma feature is used for the analysis of gene expression data arising from microarray analysis.

Given the sample size available for analysis (n=58), differentially methylated CpG for survival status showing an adjusted p-value of <0.1 were considered for inclusion in the molecular component of the prognostic panel. Heat maps were constructed using hierarchical clustering analysis using the heatmap package v1.0.12 in R employing survival status as the clustering variable. To evaluate for enrichment of differentially methylated genes among pathways, pathway analysis was conducting using two complementary and overlapping annotations: gene ontology (GO) and Kyoto Encyclopedia of Genes and Genomes (KEGG). Pathway analysis, specifically overrepresentation analysis, was pursued using KEGG. And, GO annotations was performed using clusterProfiler v3.16.1 in R, with non-significant differentially expressed genes specified as the “background universe” and accounting for multiple testing using Bonferroni correction. For overrepresentation analysis employing GO annotations, pathways were categorized further into biological process, molecular function, and cellular compartment. Differentially methylated pathways were evaluated in relation to each other and contributing differentially methylated sites by two visualizations of functional enrichment (i.e., dot plot and gene-concept networks) using the enrichplot package v1.8.1 in R.

A correlation of the expression of genes that harbored differentially methylated sites associated with survival status was developed. An analysis of gene expression collected by RNA sequencing (RNAseq) was performed from early-stage OSCC patients in the TCGA database. Raw gene counts were obtained from TCGA. Only genes with at least 10 counts in at least 90% of the sample were retained for analysis. The Ensembl identifiers (ID) of the gene counts were annotated to Entrez IDs using the EnrichmentBrowser v 2.18.2 Package in R. Annotations for the genes was given using the Homo. sapiens v.1.3.1 package. Correlation of RNAseq gene counts to CpG site methylation was performed using STATA/SE 14.2 (StataCorp, College Station, TX).

Statistical analyses were performed in STATA/SE 14.2. For each cohort, univariate analyses were performed to determine distributional characteristics and assess for randomness of the missing data (variables to be included in the final prognostic panel risk factor score had less than 5% missing values so imputation was not performed). Bivariate analyses with the primary outcome (vital status [survival vs. death] at 5-year follow-up) were performed on candidate variables (based on selection of the investigators from a detailed screening of relevant clinical and demographic risk factors) with the outcome variable. For continuous variables, cut-offs were derived using the chi-square interaction detected by manual adjustment to ensure that cut-offs made sense clinically. Recursive partitioning was used to derive a final non-molecular scoring system to predict survival status at 5-year follow-up with the goal of minimizing the number of misclassified values in the final cell while maximizing the simplicity of the score. Odds ratios at each decision node were rounded to the nearest integer to create the score. Operating characteristics of the derived risk score were calculated on both the discovery (internal cohort, n=515) and validation (TCGA, n=58) cohorts. The concordance statistic (c-index), equivalent to the area under the receiver operating curve (AUROC), was used to assess model discrimination and fit using the derived risk factor score to predict OSCC patients at risk for early mortality and morbidity. The range of the c-index is from 0.5 (random concordance) to 1 (perfect concordance).

The DNA methylation-based, molecular component of the REASON score was developed according to a methylation state transition matrix. For each of the CpG sites, a β-value of <0.3 indicated an unmethylated state, 0.33-0.75 a hemi-methylated state, and >0.75 a fully methylated state. A gene was considered to be hypermethylated if the methylation level moved from a less methylated state to a more methylated state. Conversely, a gene was considered hypomethylated if there was a state change to a lower level. A change in methylation that did not have a state change was not considered significant. The REASON score was established by combining the presence or absence of each non-molecular and molecular risk factor. The c-index was derived as described above by comparing the observed survival status at 5 years with the predicted survival status at 5 years using the individual REASON score.

Sample collection methods were developed to implement a non-invasive robust method of assessment of OSCC. Correlations were calculated between cancer and normal tissues and brush swab samples for each patient to determine the robustness of DNA methylation marks using brush swabs in clinical biomarker studies.

Three OSCC patients underwent collection of cancer and contralateral normal tissue and brush swab biopsies, totaling 4 samples for each patient. Epigenome-wide DNA methylation quantification was performed using the SureSelect^{XT} Methyl-Seq platform. DNA quality and methylation site resolution were compared between brush swab and tissue samples. The patients were enrolled in a multi-institutional prospective clinical study in which biological samples and clinicopathologic information were collected. Collection of clinical data and samples was approved by the Institutional Review Board at each institution, which included Loma Linda University (LLU), University of Illinois Chicago (UIC), and University of Alabama at Birmingham (UAB). Patients were eligible if they were ≥18 years of age, had biopsy-proven squamous cell carcinoma of oral cavity sub-sites, including oral tongue, maxillary and mandibular gingiva, hard palate, floor of mouth, buccal mucosa, and lip mucosa, and no previous treatment of OSCC. Clinical and pathologic stages were recorded based on the American Joint Committee on Cancer (AJCC) Eighth Edition Staging Manual. The following information was collected from the chart review: age, sex, race, smoking and alcohol use, staging, tumor location, pathologic characteristics, and treatment modalities received in addition to tumor ablation. Biological samples collected at the time of surgery include flash-frozen cancer and contralateral normal tissue, and brush swab biopsies of the cancer and contralateral normal site. Isohelix brush swabs (Boca Scientific) were brushed for a total of 20 times, with 10 times on each surface of the swab, at either the cancer or contralateral normal site. The brush swabs were preserved using 500 ul BuccalFix™ stabilization solution (Boca Scientific). Samples were stored in −80° C.

A total of 3 patients were randomly chosen from the ongoing prospective clinical study for the current study. DNA was extracted from the flash-frozen tissue and brush swabs of the cancer and contralateral normal side of 3 patients, totaling 12 samples (4 samples per patient). Genomic DNA quality was determined by spectrophotometry and concentration was determined by fluorometry. DNA integrity and fragment size were determined using a microfluidic chip run on an Agilent Bioanalyzer.

Indexed paired-end whole-genome sequencing libraries were prepared using the SureSelect XT Methyl-Seq kit (Agilent). Genomic DNA was sheared to a fragment length of 150-200 bp using the Covaris E220 system. Fragmented sample size distribution was determined using the Caliper LabChip GX system (PerkinElmer). Fragmented DNA ends were repaired with T4 DNA Polymerase and Polynucleotide Kinase and “A” base was added using Klenow fragment followed by AMPure XP bead-based purification (Beckman Coulter). The methylated adapters were ligated using T4 DNA ligase followed by bead purification with AMPure XP. Quality and quantity of adapter-ligated DNA were assessed with the Caliper LabChip GX system. Samples were enriched for targeted methylation sites by using the custom SureSelect Methyl-Seq Capture Library. Hybridization was performed at 65° C. for 16 h using a thermal cycler. Once the enrichment was completed, the samples were mixed with streptavidin-coated beads (Thermo Fisher Scientific) and washed with a series of buffers to remove non-specific DNA fragments. DNA fragments were eluted from beads with 0.1 M NaOH. Unmethylated C residues of enriched DNA underwent bisulfite conversion using the EZ DNA Methylation-Gold Kit (Zymo Research). The SureSelect enriched and bisulfite-converted libraries underwent PCR amplification using custom made primers (IDT). Dual-indexed libraries were quantified by quantitative polymerase chain reaction (qPCR) with the Library Quantification Kit (KAPA Biosystems) and inserts size distribution was assessed using the Caliper LabChip GX system. Samples were sequenced using 100 bp paired-end sequencing on an Illumina HiSeq NovaSeq according to Illumina protocol. A positive control (prepared bacteriophage Phi X library) was added into every lane at a concentration of 0.3% to assess sequencing quality in real time.

Signal intensities were converted to individual base calls during each run using the system's Real Time Analysis software. Sample de-multiplexing was performed using Illumina's CASAVA 1.8.2 software suite. The sample error rate was required to be less than 1% and the distribution of reads per sample in a lane to be within reasonable tolerance. Sequence data quality were examined using FastQC (ver. 0.11.8). Adapter sequences and fragments with poor quality were removed by Trim galore (ver. 0.6.3_dev). Bismark pipelines (ver. v0.22.1_dev) were used to align the reads to the bisulfite human genome (hg19) with default parameters. Sample alignment to the human genome was performed using bowtie 2 (ver. 2.3.5.1). Quality-trimmed paired-end reads were converted into a bisulfite forward (C->T conversion) or reverse (G->A conversion) strand read. Duplicated reads were removed from the Bismark mapping output and CpG extracted. All CpG sites were grouped by sequencing coverage (i.e., read depth); CpG sites with coverage ≥10× depth were retained for analysis to ensure high MC-Seq data quality. Genes were annotated using Homer annotatePeaks.pl. With this software, the promoter region is defined as 1 kilobase from the transcription start site (TSS). The Benjamini-Hochberg FDR process was applied to adjust p values per CpG site. Pearson correlations were calculated between tissue and brush biopsy samples of matched anatomic sites, and cancer and normal samples from the same patients. Pearson correlation and absolute difference were calculated among common CpG sites between the samples. Scatterplots were rendered showing the correlation of β values from all CpG sites measured by MC-seq. Separate scatterplots were rendered showing the concordance of these CpG sites between tissues and brush swabs for the cancer sites and the normal sites. Student t-tests were performed to compare β values between cancer and normal groups or tissue and brush swab groups. The most significant 1,000 CpGs features in cancer vs. normal groups were selected. Based on these results, the −log 10 (t-test p-value) was calculated for each of the 1,000 CpG sites to compare the degree of divergence in the significance of the test statistics for these 1,000 CpG between (1) cancer vs. normal and (2) tissue vs. brush swabs. Statistical analyses were performed in R environment (v. 4.1.0).

### Methylation Array Analysis Reveals Differentially Methylated Genes in Early Stage OSCC Patients Who Did not Survive to 5 Years

Of the 4,544 genes harboring CpG sites meeting criteria for analysis, 12 genes showed an adjusted p-value of <0.1 (Table 2). Gene position and methylation fold-change values are shown. The methylation trends for each gene that are predictive of poor survival here are shown, in comparison to the gene expression trends that are predictive of poor survival in previous studies. The PMID of the referenced study is included. They included ABCA2, CACNA1H, CCNJL, GPR133, HGFAC, HORMAD2, MCPH1, MYLK, RNF216, SOX8, TRPA1, and WDR86.

12 GENE METHYLATION SIGNATURE IN HIGH VS. LOW RISK GROUPS

| | | | | | | adjusted | t- | beta |
|---|---|---|---|---|---|---|---|---|
| gene | array name | chromosome | position | logFC | p value | p value | statistic | value |
| HORMAD2 | cg2 [symbol] 211826 | 22 | 3057232 [symbol] | 1.2 [symbol] | 1.27E−06 | 0.072 | [symbol] | 4.60 |
| MYLK | cg00465319 | 3 | 12 [symbol] 33 [symbol] 565 | 0.957 | 2.23E−06 | 0.072 | [symbol] .3 [symbol] | 4. [symbol] 5 |
| GPR133 | cg240 [symbol] 8252 | 12 | 131580577 | 1.3 [symbol] | 2.58E−06 | 0.072 | [symbol] .26 | 4.03 |
| SOX8 | cg021707 [symbol] 4 | 16 | 1034527 | 1.233 | 3.04E−06 | 0.072 | 6.21 | 3.90 |
| TRPA1 | cg27528 [symbol] 0 | 6 | 72 [symbol] 87924 | [symbol] | 4.11E−06 | 0.073 | [symbol] | 3.65 |
| ABCA2 | cg14280263 | [symbol] | 13 [symbol] 03861 | 2. [symbol] 26 | 4.60E−06 | 0.073 | 6.0 [symbol] | 3.56 |
| HGFAC | cg02617981, | [symbol] | 34 [symbol] 663, | 1.719 | 6.26E−06 | 0.083 | 5. [symbol] 1 | 3.31 |
| | cg12337559 | | 344 [symbol] 04 | 1.777 | 7.89E−06 | | 4.94 | 3.11 |
| MCPH1 | cg0 [symbol] 035995 | 8 | 5323 [symbol] 31 | 1.252 | 7.74E−06 | 0.083 | 4. [symbol] 5 | 3.13 |
| WDR86 | cg26438167 | 7 | 151067979 | 1.044 | 1.21E−05 | 0.093 | 4.82 | 2.77 |
| CACNA1H | cg16585866 | 16 | 1222626 | 1.056 | 1.23E−05 | 0.093 | 4. [symbol] 2 | 2.76 |
| RNF216 | cg08180025 | 7 | 5 [symbol] | 0. [symbol] 23 | 1.26E−05 | 0.093 | 4. [symbol] 1 | 2.74 |
| CCNJL | cg19100159 | 5 | 159885203 | [symbol] | 1.28E−05 | 0.093 | [symbol] | 2.73 |

| 12 GENE METHYLATION | THIS STUDY | PREVIOUSLY PUBLISHED STUDIES | | | |
|---|---|---|---|---|---|
| SIGNATURE IN HIGH | methylation | expression | | | |
| VS. LOW RISK GROUPS | TRENDS PREDICTIVE OF | | mechanism | | |
| gene | POOR SURVIVAL | | examined | cancer type | PMID |
| HORMAD2 | [symbol] | — | SNP [symbol] | NSCLC | 24797335 |
| | | — | SNP [symbol] | NSCLC | 2172530 [symbol] |
| | | [symbol] | methylation | thyroid | 30 [symbol] 914 |
| | | | expression | carcinoma | |
| MYLK | [symbol] | [symbol] | expression | bladder | 286 [symbol] 7 [symbol] 57 |
| | | | | carcinoma | |
| | | [symbol] | expression | CRC | 2 [symbol] 4 [symbol] 4 |
| | | [symbol] | expression | HCC | 3098 [symbol] 71 [symbol] |
| GPR133 | [symbol] | [symbol] | expression | GBM | 2777 [symbol] 701 |
| | | [symbol] | expression | GBM | 3264270 [symbol] |
| SOX8 | [symbol] | [symbol] | | OSCC | 29071717 |
| | | [symbol] | expression | endometrial | 3 [symbol] 1 [symbol] 05 [symbol] 7 |
| | | | | carcinoma | |
| TRPA1 | [symbol] | [symbol] | expression | nasopharyngeal | 273 [symbol] 05 [symbol] 2 |
| | | | | carcinoma | |
| | | [symbol] | expression | renal clear cell | 2670 [symbol] 03 |
| | | | | carcinoma | |
| ABCA2 | [symbol] | [symbol] | expression | EOC | 26 [symbol] 2 [symbol] 484 |
| | | [symbol] | expression | ALL | 24145140 |
| HGFAC | [symbol] | [symbol] | methylation | HCC | 30 [symbol] 3594 [symbol] |
| | | | expression | | |
| | | [symbol] | expression | EOC | 11454421 |
| MCPH1 | [symbol] | [symbol] | expression | breast ductal | 20 [symbol] 86 |
| | | | | carcinoma | |
| | | [symbol] | expression | ovarian | 21505456 |
| | | | | carcinoma | |
| WDR86 | [symbol] | [symbol] | expression | CRC | 3240 [symbol] 88 |
| | | [symbol] | expression | breast | 325397 [symbol] 2 |
| | | | | carcinoma | |
| CACNA1H | [symbol] | [symbol] | expression | gastric, lung, | 2864 [symbol] 97 |
| | | | | ovarian | |
| RNF216 | [symbol] | [symbol] | expression | CRC | 27203 [symbol] 74 |
| | | [symbol] | expression | ovarian | 28 [symbol] 0 |
| | | | | carcinoma | |
| CCNJL | [symbol] | [symbol] | expression | HCC | 332045 [symbol] 4 |

ALL—acute lymphoblastic leukemia;
CRC—colorectal carcinoma;
EOC—epithelial ovarian carcinoma;
GBM—glioblastoma multiforme;
HCC—hepatocellular carcinoma;
NSCLC—non small cell lung carcinoma;
OSCC—oral squamous cell carcinoma [symbol]
indicates data missing or illegible when filed

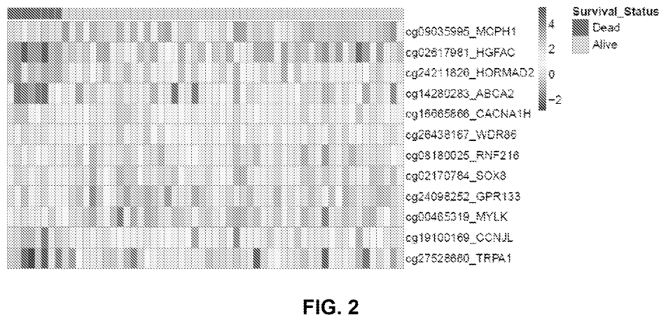

FIG. 2 is a heat map and hierarchical clustering of differentially methylated genes demonstrates distinct methylation signature in high-risk vs. low-risk OSCC patients. FIG. 2 illustrates the methylation state for each of the 58 TCGA patient samples of the 12 top differentially methylated genes using a heat map. Patients who died by 5 years due to their cancer are grouped on the left of the heat map, with significant differences in methylation signatures compared to patients who survived to 5 years.

A literature search of each of the 12 genes revealed that with the exception of SOX8, none of the genes had previously been linked to OSCC in either human or preclinical studies. In Table 2 each of the genes is linked to the referenced clinical studies demonstrating poor cancer survival. HORMAD2 dysregulation through either SNPs or hypermethylation is attributed to poor survival in non-small cell lung cancer (NSCLC) and thyroid carcinoma. MYLK over-expression is linked to poor survival in bladder carcinoma, colorectal carcinoma, and hepatocellular carcinoma. GPR133 expression is inversely correlated with survival in patients with glioblastoma multiforme. The role of SOX8 has been already been investigated using in vitro models and in vivo models, as well as in clinical samples of OSCC. In a clinical study, SOX8 is over-expressed in chemoresistant patients with tongue SCC and is associated with higher lymph node metastasis, advanced tumor stage, and shorter overall survival. Similarly, higher SOX8 expression is linked to a high tumor histological grade, lymph node metastasis, and shorter overall survival in patients with endometrial carcinoma. TRPA1 expression in cancer is controversial, with gene over-expression linked to poor survival in nasopharyngeal carcinoma and gene under-expression linked to poor survival in renal clear cell carcinoma. However, a study using International Cancer Genome Consortium data shows that the TRP family of genes has varying expression across different cancer types, and that some TRP genes have stronger prognostic ability than others. ABCA2, which encodes for a membrane-associated protein of the superfamily of ATP-binding cassette transporters, is over-expressed in epithelial ovarian carcinoma and acute lymphoblastic leukemia patients with poor survival. HGFAC expression is directly correlated to survival in breast ductal carcinoma and ovarian carcinoma. WDR86 expression is linked to poor survival in colorectal carcinoma and breast carcinoma. In a clinical study of solid tumors including gastric, lung and ovarian cancer, expression of T-type calcium channel genes including CACNA1H is used as a prognostic signature for survival. RNF216 expression is associated with poor survival in colorectal cancer and ovarian carcinoma, although whether over- or under-expression decreases survival is unknown. CCNJL expression is inversely correlated with survival in hepatocellular carcinoma.

Of note, differential methylation of the 12 genes has not previously been linked to poor survival in any type of cancer. With the exception of HORMAD2 and HGFAC, published studies on these candidate genes have focused on differential gene expression rather than methylation.

### Prognostic Ability of the REASON Score

The REASON score was calculated by combining the 10-factor non-molecular panel with the 12-gene methylation panel composed of 13 CpGs, in which methylation status of each gene was determined using the methylation state transition matrix. The REASON score predicted 5-year disease-specific mortality with a c-index=0.915.

### Functional Analysis of the Differentially Methylated Genes

Gene expression data was available for 55 of the 58 TCGA OSCC patients with DNA methylation data. As is becoming increasingly appreciated, gene hypermethylation can result in decreased or increased gene expression, which was observed in the TCGA sample. Significant correlation between gene expression and DNA methylation at each gene was observed for 6 (ABCA2 [r=0.46, p=0.0005], GPR133 [r=0.42, p=0.0015], MCPH1 [r=0.31, p=0.024], RNF216 [r=−0.38, p=0.0045], TRPA1 [r=−0.60, p<0.0001], WDR86 [r=0.36, p=0.0072]) of the 12 genes. Additionally, gene network analysis was performed through the publicly available databases to determine whether the 12 candidate genes were directly involved in established signaling networks. Table 3 details the KEGG pathways that are linked to the candidate genes.

TABLE 3
Functional Network Analysis (KEGG)

| ID | Description | p_{UNADJUSTED} | p_{BONFERRONI} | q-value | Top 12 Genes |
|---|---|---|---|---|---|
| hsa04020 | Calcium signaling pathway | 3.43E−12 | 1.12E−09 | 5.27E−10 | CACNA1H, MYLK |
| hsa04080 | Neuroactive ligand-receptor interaction | 4.12E−12 | 1.34E−09 | 5.27E−10 | |
| hsa05032 | Morphine addiction | 2.24E−10 | 7.28E−08 | 1.91E−08 | |
| hsa04360 | Axon guidance | 1.48E−08 | 4.809E−06 | 8.01E−07 | |
| hsa04510 | Focal adhesion | 1.57E−08 | 5.09E−06 | 8.01E−07 | MYLK |
| hsa05033 | Nicotine addiction | 8.67E−08 | 2.819E−05 | 3.698E−06 | |
| hsa04015 | Rap1 signaling pathway | 2.77E−07 | 9.009E−05 | 1.013E−05 | |
| hsa04024 | cAMP signaling pathway | 3.65E−07 | 0.0001185 | 1.166E−05 | |
| hsa04724 | Glutamatergic synapse | 5.50E−07 | 0.0001788 | 1.564E−05 | |
| hsa05412 | Arrhythmogenic right ventricular cardiomyopathy | 4.518E−06 | 0.0014683 | 0.0001158 | |
| hsa04725 | Cholinergic synapse | 7.459E−06 | 0.0024243 | 0.0001735 | |
| hsa04713 | Circadian entrainment | 1.151E−05 | 0.0037401 | 0.000228 | CACNA1H |
| hsa04810 | Regulation of actin cytoskeleton | 1.159E−05 | 0.0037655 | 0.000228 | MYLK |
| hsa05200 | Pathways in cancer | 1.463E−05 | 0.0047544 | 0.0002673 | |
| hsa04151 | Pl3K-Akt signaling pathway | 2.039E−05 | 0.0066282 | 0.0003478 | |
| hsa04974 | Protein digestion and absorption | 2.177E−05 | 0.0070739 | 0.000348 | |
| hsa04727 | GABAergic synapse | 3.33E−05 | 0.0108238 | 0.0005011 | |
| hsa04925 | Aldosterone synthesis and secretion | 4.05E−05 | 0.0131628 | 0.0005755 | CACNA1H |
| hsa04014 | Ras signaling pathway | 4.699E−05 | 0.0152703 | 0.0006325 | |

Table 4 details the GO pathways that are linked to the candidate genes aggregated by gene ontology category (i.e., biological process, cellular compartment, molecular function). Differentially methylated pathways (adjusted p-value<0.05) based on GO annotations are shown. Differentially methylated pathways were evaluated based on Biological Process (BP), Molecular Function (MF), and Cellular Compartment (CC) ontologies. Pathways that include any of the 12 differentially methylated genes included in the prognostic panel are identified.

| p_{UNADJUSTED}- | p_{BONFERRONI}- | | | |
|---|---|---|---|---|
| value | value | q-value | Ont | Top 12 Genes |
| 4.56E−16 | 2.76E−12 | 4.40E−13 | BP | RNF216 |
| 7.67E−16 | 4.63E−12 | 5.32E−13 | BP | MCPH1 |
| 7.72E−16 | 4.66E−12 | 5.32E−13 | BP | RNF216 |
| 6.87E−14 | 4.15E−10 | 3.02E−11 | BP | SOX8 |
| 1.26E−13 | 7.58E−10 | 5.05E−11 | BP | CACNA1H |
| 3.84E−13 | 2.32E−09 | 1.42E−10 | BP | CACNA1H |
| 1.82E−11 | 1.10E−07 | 5.16E−09 | BP | MCPH1 |
| 2.22E−10 | 1.3414E−06 | 4.56E−08 | BP | MCPH1 |
| 1.70E−09 | 1.0265E−05 | 2.65E−07 | BP | SOX8 |
| 4.40E−09 | 2.6566E−05 | 6.43E−07 | BP | SOX8 |
| 6.05E−09 | 3.6548E−05 | 8.59E−07 | BP | CACNA1H, MYLK, TRPA1 |
| 6.61E−09 | 3.9941E−05 | 8.87E−07 | BP | MYLK |
| 6.61E−09 | 3.9941E−05 | 8.87E−07 | BP | SOX8 |
| 6.96E−09 | 4.2015E−05 | 9.07E−07 | BP | TRPA1 |
| 8.92E−09 | 5.3868E−05 | 1.1327E−06 | BP | SOX8 |
| 1.48E−08 | 8.9603E−05 | 1.5234E−06 | BP | SOX8 |
| 1.55E−08 | 9.3436E−05 | 1.5362E−06 | BP | SOX8 |
| 2.16E−08 | 0.0001304 | 2.0594E−06 | BP | SOX8 |
| 2.22E−08 | 0.00013411 | 2.0608E−06 | BP | MYLK, SOX8 |
| 2.48E−08 | 0.00014994 | 2.2187E−06 | BP | MCPH1 |
| 2.78E−08 | 0.00016787 | 2.3854E−06 | BP | CACNA1H, TRPA1 |
| 2.98E−08 | 0.00017993 | 2.4788E−06 | BP | SOX8 |
| 4.11E−08 | 0.0002483 | 3.2001E−06 | BP | SOX8 |
| 6.99E−08 | 0.00042223 | 4.9009E−06 | BP | CACNA1H, SOX8 |
| 8.07E−08 | 0.00048726 | 5.5622E−06 | BP | MYLK, SOX8 |
| 8.78E−08 | 0.00053061 | 5.9717E−06 | BP | CACNA1H, MYLK, TRPA1 |
| 1.55E−07 | 0.00093504 | 9.697E−06 | BP | CACNA1H |
| 1.58E−07 | 0.00095203 | 9.697E−06 | BP | SOX8 |
| 1.79E−07 | 0.00108306 | 1.0684E−05 | BP | CACNA1H, MYLK, TRPA1 |
| 2.66E−07 | 0.00160841 | 1.512E−05 | BP | SOX8 |
| 2.86E−07 | 0.00172748 | 1.5866E−05 | BP | SOX8 |
| 3.82E−07 | 0.00230961 | 2.0506E−05 | BP | CACNA1H |
| 4.32E−07 | 0.00260965 | 2.2915E−05 | BP | CACNA1H |
| 7.14E−07 | 0.00431029 | 3.7034E−05 | BP | TRPA1 |
| 8.00E−07 | 0.00483277 | 3.9123E−05 | BP | MYLK |
| 8.67E−07 | 0.00523964 | 4.1453E−05 | BP | TRPA1 |
| 8.81E−07 | 0.00532278 | 4.1698E−05 | BP | CACNA1H, MYLK |
| 1.2018E−06 | 0.00725879 | 5.524E−05 | BP | TRPA1 |
| 1.2803E−06 | 0.00773314 | 5.775E−05 | BP | CACNA1H, MYLK, SOX8 |
| 1.509E−06 | 0.00911415 | 6.6207E−05 | BP | SOX8 |
| 1.5452E−06 | 0.0093333 | 6.7188E−05 | BP | CACNA1H |
| 1.563E−06 | 0.00944082 | 6.7355E−05 | BP | MYLK |
| 1.5956E−06 | 0.00963749 | 6.815E−05 | BP | CACNA1H |
| 1.7143E−06 | 0.01035458 | 7.1327E−05 | BP | SOX8 |
| 1.9635E−06 | 0.01185943 | 8.0528E−05 | BP | SOX8 |
| 2.8198E−06 | 0.01703131 | 0.0001055 | BP | CACNA1H, MYLK |
| 2.8795E−06 | 0.01739194 | 0.00010609 | BP | CACNA1H, MYLK |
| 3.1078E−06 | 0.01877121 | 0.00011363 | BP | SOX8 |
| 4.4201E−06 | 0.02669767 | 0.00015459 | BP | TRPA1 |
| 4.6137E−06 | 0.02786667 | 0.0001585 | BP | MCPH1 |
| 5.2667E−06 | 0.03181087 | 0.00017652 | BP | SOX8 |
| 5.5471E−06 | 0.03350478 | 0.00018337 | BP | CACNA1H |
| 5.7637E−06 | 0.03481246 | 0.00018795 | BP | SOX8 |
| 5.998E−06 | 0.03622772 | 0.00019428 | BP | SOX8 |
| 6.9375E−06 | 0.04190233 | 0.00021884 | BP | MYLK |
| 7.294E−06 | 0.04405592 | 0.00022422 | BP | CACNA1H, MYLK |
| 7.3464E−06 | 0.04437238 | 0.00022441 | BP | SOX8 |
| 2.29E−17 | 1.59E−14 | 6.32E−15 | CC | ABCA2, CACNA1H |
| 8.75E−17 | 6.09E−14 | 1.44E−14 | CC | CACNA1H |
| 1.30E−16 | 9.08E−14 | 1.44E−14 | CC | ABCA2, CACNA1H |
| 4.44E−16 | 3.09E−13 | 4.09E−14 | CC | CACNA1H |
| 1.58E−10 | 1.10E−07 | 6.24E−09 | CC | RNF216 |
| 1.69E−08 | 1.1791E−05 | 4.07E−07 | CC | MYLK |
| 1.9183E−06 | 0.00133514 | 2.7898E−05 | CC | MYLK |
| 1.7515E−05 | 0.01219037 | 0.00019754 | CC | CACNA1H |
| 1.8407E−05 | 0.01281115 | 0.00020344 | CC | RNF216 |
| 1.54E−15 | 1.65E−12 | 1.41E−12 | MF | TRPA1, CACNA1H |
| 3.67E−15 | 3.93E−12 | 1.69E−12 | MF | TRPA1, CACNA1H |
| 3.13E−14 | 3.36E−11 | 9.36E−12 | MF | TRPA1, CACNA1H |
| 4.07E−14 | 4.36E−11 | 9.36E−12 | MF | TRPA1, CACNA1H |
| 2.28E−13 | 2.44E−10 | 4.20E−11 | MF | TRPA1, CACNA1H |
| 3.00E−13 | 3.22E−10 | 4.61E−11 | MF | TRPA1, CACNA1H |
| 1.14E−10 | 1.23E−07 | 1.32E−08 | MF | CACNA1H |
| 1.14E−10 | 1.23E−07 | 1.32E−08 | MF | CACNA1H |
| 2.06E−10 | 2.21E−07 | 2.11E−08 | MF | SOX8 |
| 2.61E−10 | 2.80E−07 | 2.40E−08 | MF | SOX8 |
| 7.42E−10 | 7.96E−07 | 6.21E−08 | MF | CACNA1H |
| 2.19E−08 | 2.3453E−05 | 1.2383E−06 | MF | CACNA1H, TRPA1 |
| 2.60E−07 | 0.00027872 | 1.0875E−05 | MF | CACNA1H, TRPA1 |
| 9.43E−07 | 0.00101226 | 3.4756E−05 | MF | MYLK |
| 1.0407E−05 | 0.01116647 | 0.00029046 | MF | TRPA1 |
| 1.0407E−05 | 0.01116647 | 0.00029046 | MF | TRPA1 |
| 1.2816E−05 | 0.01375127 | 0.00034718 | MF | MYLK |

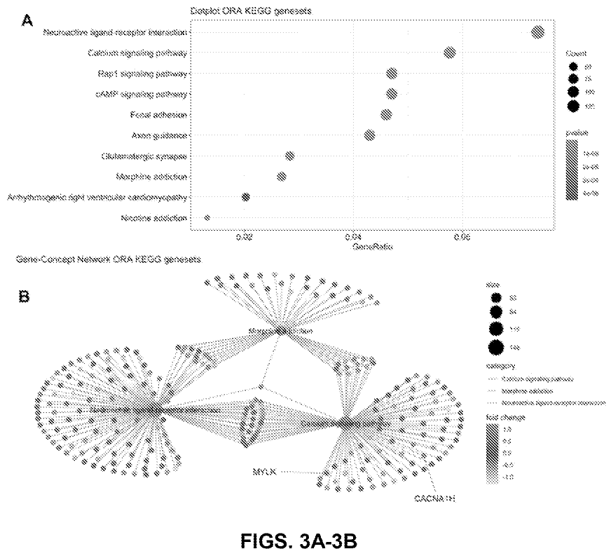

Seven of the 12 differentially methylated genes (i.e., ABCA2, CACNA1H, MCPH1, MYLK, RNF216, SOX8, TRPA1) mapped to statistically significant differentially methylated pathways. The complex associations between differentially methylated genes mapped to multiple related differentially methylated pathways were visualized using a geneset enrichment dotplot and a gene-concept network plot (FIGS. 3A and 3B). FIGS. 3A and 3B are representations from functional network analysis mapping. Functional enrichment analysis identifies the aggregation of differentially methylated genes ontp pathways that aggregate to three concepts. FIG. 3A is a Dot plot of differentially enriched genes that map to the top ten most differentially perturbed methylated pathways (p_{adjusted}<0.05). FIG. 3B is a diagrammatic representation of the top 3 most statistically differentially methylated pathways are identified by a circle in grey and the fold change in differential methylation of component genes is rendered in color ranging from negative (green) to positive (red) fold change for each gene. The size of each circle is based on the number of genes.

CACNA1H and MYLK mapped to 5 of the 19 statistically differentially methylated pathways (p_{adjusted}<0.05; Table 3). These two (CACNA1H, MYLK) of the twelve differentially methylated genes included in the REASON classifier map to the top 3 most differentially methylated pathways: neuroactive ligand-receptor interaction, morphine addiction, and calcium signaling pathways

### The REASON Score has High Accuracy in Predicting Poor Survival of Early-Stage OSCC.

The REASON score is dependent on non-molecular clinicopathologic factors as well as a 12-gene methylation signature. Previous methylation studies in OSCC have not identified any of these 12 genes as indicative of the prognosis of OSCC. With the exception of SOX8, the genes within the panel have not previously been associated with OSCC. However, while some of these genes have not been firmly established as playing crucial roles in carcinogenesis, all 12 genes are linked to other cancer survival in genetic association studies on patient tissues. But, the expression profiles do not align with the methylation profile that is predictive for OSCC.

Clinicopathologic information for the 3 enrolled patients are detailed in Tables 4a and 4b. The 3 patients comprised both early and late stage OSCC (stage I and IV), as well as varying tobacco and alcohol consumption habits. Patients were 49 and 68 years old. Two patients were male and one was female. All patients were white, non-Hispanic.

TABLE 4a
Patient demographic characteristics.

| | | | | Tobacco use, | Alcohol, |
|---|---|---|---|---|---|
| Patient | Age | Sex | Race | pack years | drinks/wk |
| 1 | 68 | F | White | Never | Never |
| 2 | 68 | M | White | Former, 53 | Former, 24 |
| 3 | 49 | M | White | Current, 72 | Current, 14 |

TABLE 4b
Patient demographic characteristics (Continued).

| Patient | Site | TNM | Stage | Grade |
|---|---|---|---|---|
| 1 | Tongue | T1N0M0 | I | Moderate |
| 2 | Tongue | T4aN0M0 | IV | Moderate |
| 3 | Mandible | T4bN3bM0 | IV | Moderate |

Tables 4a and 4b provide the demographic and clinicopathologic information for the 3 patients. Abbreviations: F=female; M=male; TNM=tumor, nodes, metastases classification.

Cancer and contralateral normal tissue and brush swab biopsies collected at the time of surgery underwent DNA extraction, with the yield and quality shown in Table 5. With a total input volume of 30 μL for each sample, total input for tissue DNA ranged from 187 ng to 660 ng, and an average of 390 ng. Total input for swab DNA ranged from 51 ng to 1998 ng, with an average of 532 ng. The input range was consistent with the results demonstrating reproducible CpG site quantification using MC-Seq across this range. As shown here, DNA quantity as low as 150-300 ng and DNA quality comparable to the findings in Table 5 were successfully amplified using the methods described herein. Table 5 provides the characteristics of genomic DNA that was used as input for sequencing of tissue and brush swab biopsies.

TABLE 5
DNA quantification.

| | DNA | | | | | gDNA |
|---|---|---|---|---|---|---|
| | concentration | | | | | input, |
| Sample | ng/μl | A260 | A280 | 260/280 | 260/230 | ng |
| 1C swab | 3.96 | 0.01 | −0.004 | −2.99 | −0.18 | 118.8 |
| 1C tissue | 22.00 | 4.45 | 2.210 | 2.01 | 2.18 | 660.0 |
| 1N swab | 1.70 | −0.05 | −0.048 | 0.95 | 0.33 | 51.0 |
| 1N tissue | 6.24 | 0.44 | 0.220 | 2.01 | 2.75 | 187.2 |
| 2C swab | 4.24 | 0.01 | −0.027 | −0.27 | −0.09 | 127.2 |
| 2C tissue | 11.40 | 1.00 | 0.463 | 2.16 | 2.85 | 342.0 |
| 2N swab | 6.32 | 0.07 | 0.014 | 5.14 | −1.45 | 189.6 |
| 2N tissue | 8.00 | 0.62 | 0.290 | 2.12 | 3.04 | 240.0 |
| 3C swab | 66.60 | 1.82 | 0.971 | 1.87 | 2.86 | 1998.0 |
| 3C tissue | 21.80 | 2.16 | 1.314 | 1.65 | 0.80 | 654.0 |
| 3N swab | 23.60 | 0.45 | 0.217 | 2.06 | 4.70 | 708.0 |
| 3N tissue | 8.48 | 0.39 | 0.191 | 2.05 | 5.66 | 254.4 |

The DNA concentration and quality as assessed by spectrophotometry and fluorometry, and total DNA input for each sample, are shown in Table 5. C=cancer, N=normal.

### Mc-Seq Mapping Efficiency Assessment

Table 6 details the mapping efficiency for each biological sample. Using MC-Seq sequences mapped to the reference genome with an average mapping efficiency of 90% across all samples. MC-Seq results for each sample are shown in Table 6. The last two rows represent the average values for all swab samples and all tissue samples, respectively. C=cancer, N=normal.

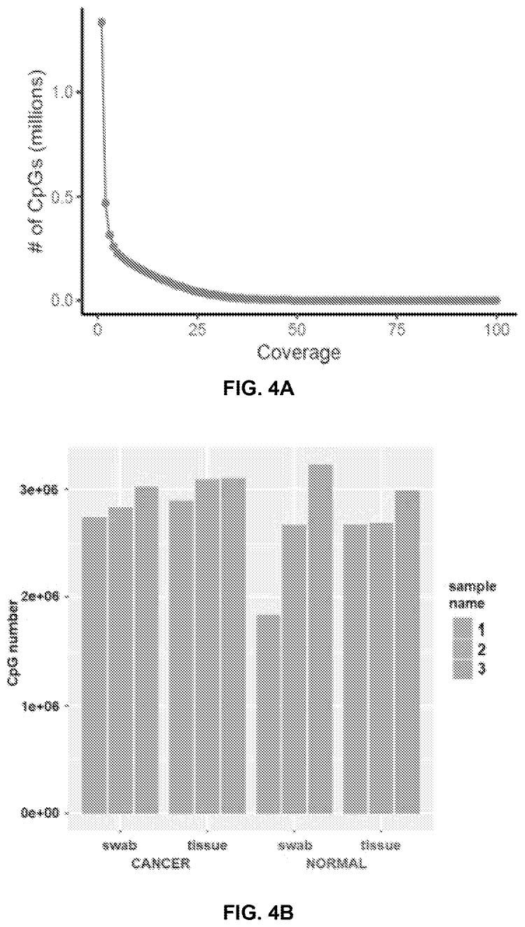

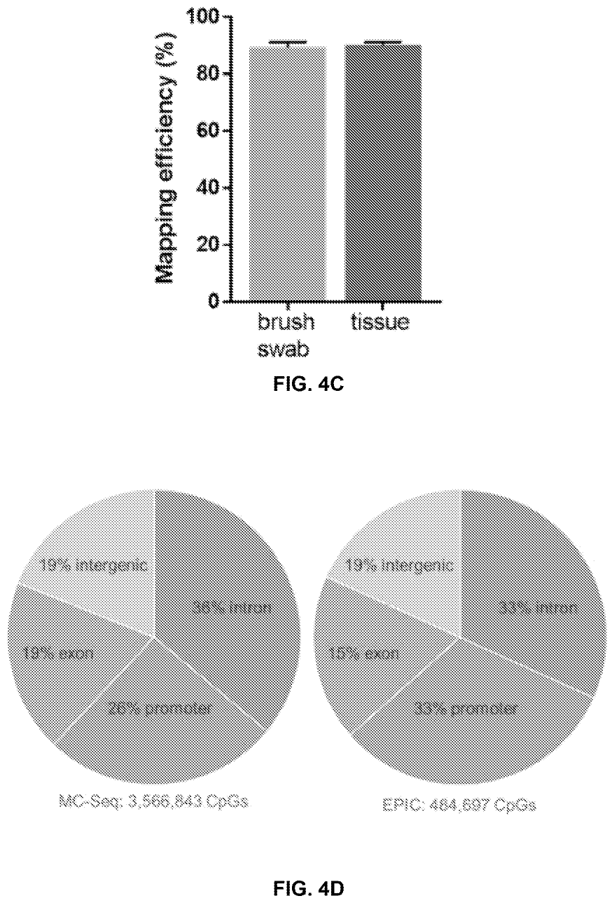

FIG. 4A is a graphical representation of the coverage in all CpGs that demonstrates an inflection point at 10× coverage. There were no significant differences in mapping efficiency between tissues and brush swab samples (FIG. 4A). FIG. 4B is a graphical representation of the number of quantified CpGs in both swab and tissue samples of cancer and normal subjects. Using 10× read depth as a cutoff, the number of quantified CpG sites was determined in each sample. The average difference in mapping efficiency between the paired brush swabs and tissues was minimal, at −0.567%, in favor of tissue samples, with a range of −1.9 to 1.7%. The majority of methylated C's appeared in a CpG context. The depth of read for each CpG was graphed across all queried CpGs and an inflection point at 10× coverage was demonstrated (FIG. 4B). These results were similar to previously provided data, in which the majority of CpG sites exhibited at least 10× coverage. This cutoff was applied, focusing the analysis on CpG sites with at least 10× coverage. Average number of CpGs with at least 10× coverage was 2,716,674 for swab samples and 2,904,261 for tissue samples, with no significant difference between the two sample types, which is in excess of 3-fold greater CpGs interrogated than the most commonly used tool to measure the DNA methylome, the Illumina EPIC array. FIG. 4C is a graphical representation of the average mapping efficiency for brush swabs and for tissues. FIG. 4C indicates the number of CpGs with at least 10× coverage for each of the 12 individual samples. The average mapping efficiency was 89.45% for brush swabs and 90% for tissues, with no significant difference between the two sampling methods.

### Distribution of Methylome Regions

The distribution of CpG sites profiled by MC-Seq was determined among the CpG sites successfully measured at 10× depth of read or greater overlapping across all 12 samples (3,566,843 CpGs).

FIG. 4D is a set of pie chart representations of the relative genic locations of the CpGs profiled by MC-Seq (left) and CpGs covered by the EPIC array that were profiled (right). MC-Seq provided more robust coverage of functional gene regions than the EPIC array. FIG. 4D demonstrates that 36% were in introns, 26% were in promoters, 19% were in exons, and 19% were in intergenic regions. Overall, MC-Seq provided more robust coverage of functional gene regions in the methylome than typically provided by the EPIC array, detecting ten-fold more CpG sites in promoter regions and exons than the EPIC array. Among the 484,697 CpGs from the EPIC array, the majority of which were also found on the 450K (396,409 CpG) were profiled by MC-Seq with at least 10× coverage. While the breakdown of these CpGs was 33% intron, 33% promoter, 15% exon, and 19% intergenic, the total number of CpGs in the functional gene regions was proportionally lower owing to the more limited coverage (FIG. 4D).

Correlation Between Brush Swab and Tissue Biopsies from Matched Anatomic Sites

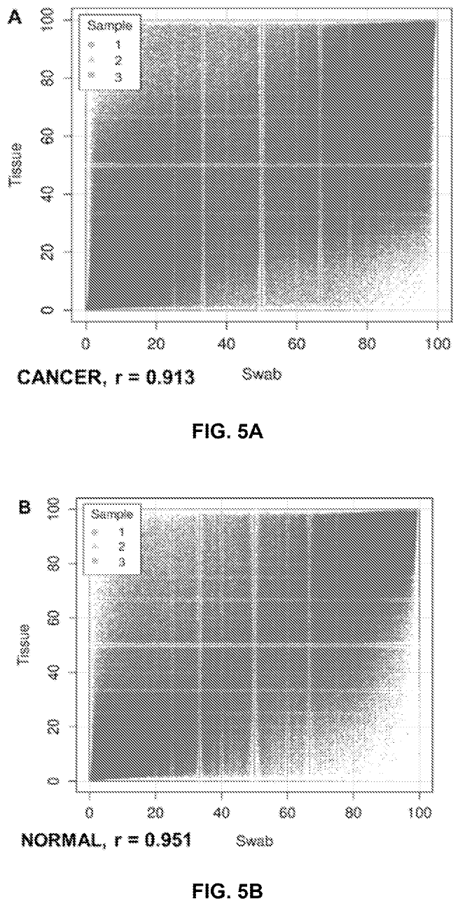

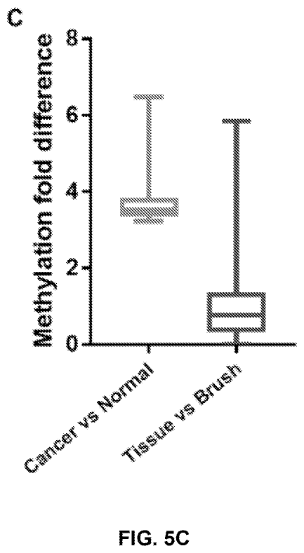

Overall, the correlation among CpG site methylation across all samples was high, all exceeding 90%. The average correlation between tissue and brush swabs (n=12) among all CpG sites shared among the entire sample (cancer+control) (s=3,566,843) was 93.2% (95% confidence interval: 93.23%, 93.25%). The average correlation between tissue and brush swabs (n=6) among all CpG sites shared among cancer samples was 91.3% (95% confidence interval: 91.32%, 91.35%). The average correlation between tissue and brush swabs (n=6) among all CpG sites shared among normal samples was 95.1% (95% confidence interval: 95.13%, 95.14%). FIGS. 5A and 5B are scatterplots demonstrating the correlation between tissue and brush swab biopsies for cancer and normal sites, respectively, of the 3 patients. The correlation values are noted. This scatterplot of the CpGs with 10× coverage demonstrated high concordance between tissue and brush swabs (FIG. 5A and FIG. 5B).

### The Top Methylation Features are Differentially Methylated Between Cancer and Normal Samples, but not Between Tissues and Brush Swabs

The top 1,000 most variable methylation features between cancer and normal samples were the focus of the analysis, which would be expected to differ considerably less between tissue and brush swab sampling methods. FIG. 5C is a graphical representation of the methylation difference between cancer and normal samples quantified with MC-Seq, visualized using box plots (median, quartiles, maximum and minimum whiskers). The p-values for each test of difference in CpG methylation by t-test were expressed as −log₁₀ (p-value), and averaged 3.67 (i.e., p=0.00021) between cancer vs. normal. The same CpG sites were not differentially methylated, with an average-log 10 (p-value)=0.96 (i.e., p=0.11) between tissue vs. brush swabs (FIG. 5C). The results suggest that brush swabs are a clinically viable surrogate for tissue biopsies.

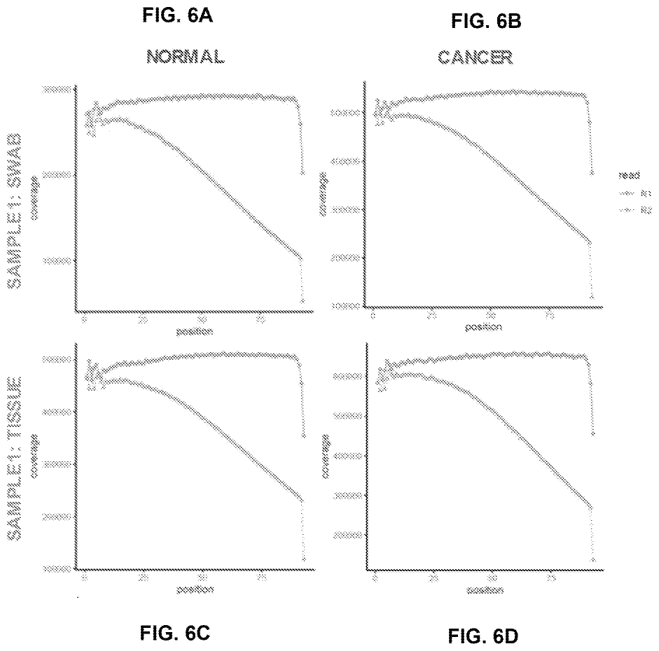

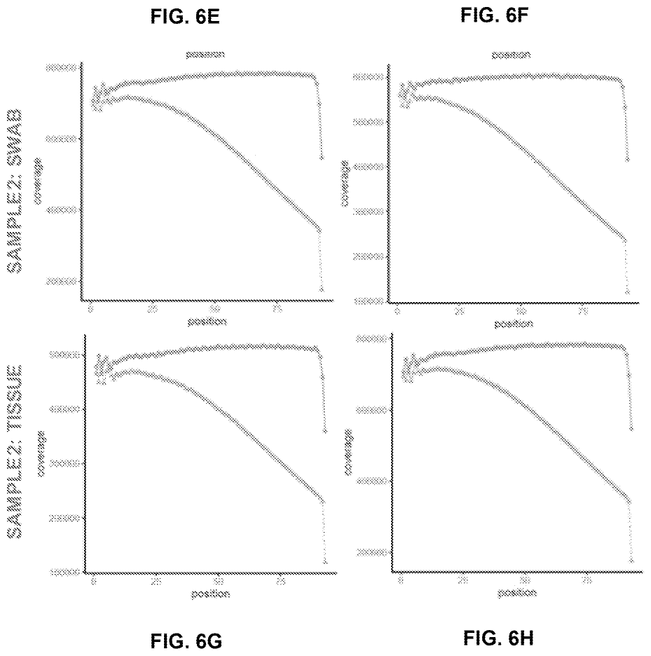

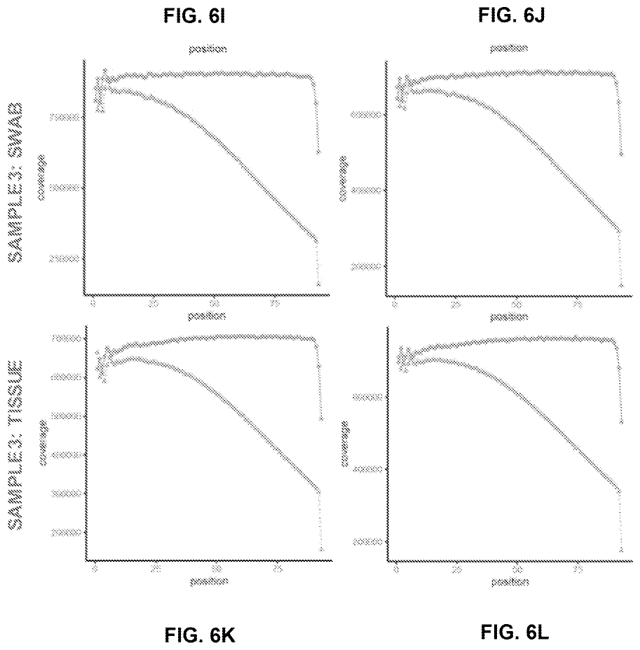

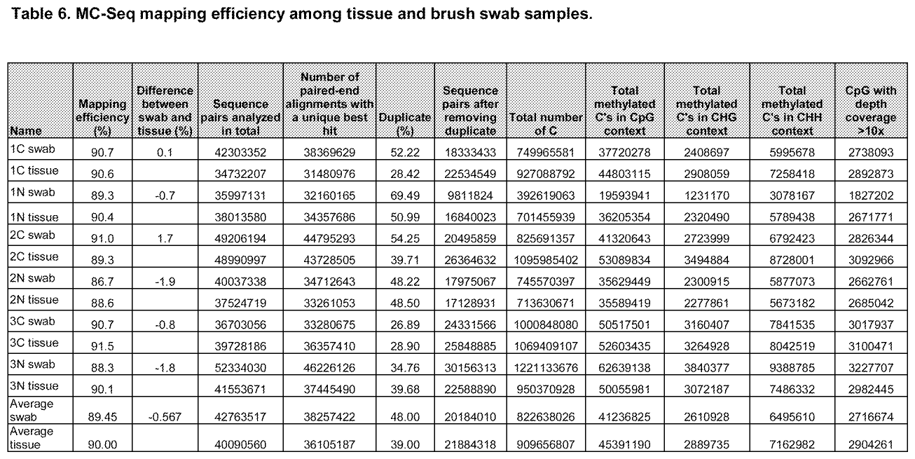

M-value bias is a standard qualitative diagnostic of the method employed to measure DNA methylation. M-value bias is examined as a function of the DNA strand that is sequenced (R1 is the “forward” strand and R2 is the same sequence but from the “reverse” strand). M-value bias has a characteristic profile where the RI strand shows high sequencing coverage for the majority of the strand while the coverage is lower and decays faster from the reverse strand. FIGS. 6A-6L are representative M-bias coverage plots demonstrating that the characteristic M-value bias is consistent in cancer samples as compared to normal samples as well as brush swab as compared to tissue biopsy. The ses of four panels (FIGS. 6A-6D, FIGS. 6E-6H, and FIGS. 6I-6L) for each of the samples (Sample 1, Sample 2, and Sample 3) are essentially identical. These data demonstrate that the source of DNA and the pathologic status of the sample does not influence M-value bias and, by inference, the quality of DNA methylation data collected.

EWAS studies in cancer patients have identified interindividual variability in the epigenome, and the recent availability of affordable EWAS technologies have led to a rapid increase in epigenetic biomarker studies aimed at identifying differential methylation features that could be predictive of clinical outcome. The most commonly used platforms are array-based, like the Illumina Human 450K and Infinium MethylationEPIC arrays, which provide limited coverage of CpG sites across the epigenome. Whole genome bisulfite sequencing (WGBS) is the most comprehensive method for epigenome profiling, capturing 28 million CpGs. However, the cost, intensive workflow, and need for high quality and quantity of DNA input significantly limit its clinical translatability, particularly in cancer treatment. MC-Seq has emerged as a promising intermediary between arrays and WGBS, using NGS to capture significantly more CpGs than array-based platforms, while having the advantage of being more high-throughput and affordable than WGBS. As shown here, MC-Seq is a more reliable and efficient platform for epigenome profiling than array-based platforms like the EPIC array. When the EPIC array and MC-Seq were compared in peripheral blood mononuclear cell samples, MC-Seq captured significantly more CpGs in coding regions and CpG islands than the EPIC array. The EPIC array captured 846,464 CpG sites per sample, whereas MC-Seq captured 3,708,550 CpG sites per sample. Of the 472,540 CpG sites captured by both platforms, there was high correlation (r=0.98-0.99) in methylation status. Moreover, while the EPIC array is enriched for genes with known roles in carcinogenesis, MC-Seq quantifies methylation in a more agnostic manner and profiles 3-4 times more CpGs than the EPIC array, allowing for a higher chance of discovering novel epigenetic modifications in cancer. Furthermore, the coverage areas within each gene were more comprehensive than the EPIC array and other commonly used methylation analysis techniques, like PCR or pyrosequencing. Disclosed here are methods involving MC-Seq that captured significantly more CpG sites within functional gene regions, owing to the higher overall profiling capability of this technique. The high throughput capabilities and depth of coverage make MC-Seq an appropriate, CLIA-approvable (Clinical Laboratory Improvement Amendments) platform to be used in a clinical setting.

Clinical translation of these methylation biomarker studies has been limited due to: 1) combining OSCC with other head and neck cancer sub-sites (i.e., oropharynyx, hypopharynx, larynx), which creates a heterogeneous cohort that fails to recognize OSCC as a distinct clinical disease, and 2) relying solely on array-based platforms, which query a limited number of CpGs. As a result, none of these studies have produced a methylation biomarker with high prognostic performance. Methylation signatures combined with clinicopathologic data were used to develop a risk score to predict 5-year mortality of early-stage (I/II) OSCC; the risk score accurately predicted mortality with a c-statistic=0.915. The REASON score leveraged the top 12 differentially methylated genes between early-stage OSCC patients who survived vs. died at 5 years after diagnosis. The differential methylation of these specific genes were correlated with outcomes in OSCC.

In addition to being a distinct clinical subsite from other head and neck sites, the oral cavity is an easily accessible anatomic site for non-invasive biopsy techniques. Clinical translation of a biomarker requires that it can be measured during treatment. Waiting until after tumor removal for the formalin-fixed, paraffin-embedded (FFPE) tissues delays potentially necessary treatment. Both saliva and brush swabs can be used to noninvasively sample OSCC cells at the time of diagnosis. Saliva has been used as a biological sample to identify methylation biomarkers of OSCC. However, concordance of methylation between saliva and cancer tissue is highly variable.

Embodiments disclosed here include methods of assessment for OSCC using brush swabs and MC-Seq to determine the methylation signature at the time of diagnosis. Brush swab and tissue biopsies from matched sites had highly correlated methylation signatures. The DNA quality and quantity from brush swab samples were adequate to perform MC-Seq. Mapping efficiency was equivalent between tissues and brush swabs. Given the high correlation between the paired tissues and brush swabs, and the satisfactory DNA yield, brush swabs serve as a clinically robust surrogate to tissue biopsies. MC-Seq offered broader coverage of CpG sites and that sample-based correlation was high (r=0.98) between the two platforms. Thus, collection of brush swabs is a noninvasive method to determine methylation signatures for risk stratification.

Oral cancer survival has not improved in the past four decades. In fact, worldwide OSCC incidence is on the rise. In an epidemiologic study of 22 cancer registries worldwide, tongue cancer incidence has increased in young women <45 years old without traditional risk factors of tobacco or alcohol use. OSCC is not caused by human papillomavirus (HPV), unlike oropharyngeal SCC, in which the majority of newly diagnosed cases are associated with HPV positivity. HPV-positive oropharyngeal SCC has significantly better survival than HPV-negative disease, with a three-year overall survival of 82.4% compared to just 57.1% for the HPV-negative group in the retrospective analysis of the Radiation Therapy Oncology Group (RTOG) 0129 trial. Overall survival of HPV-positive oropharyngeal SCC has increased to 90% with clinical trials targeting this specific disease subset. Similarly, the introduction of immunotherapy as a fourth treatment modality in head and neck SCC following FDA approval of nivolumab, a programmed cell death protein 1 (PD-1) inhibitor, and pembrolizumab, a programmed death-ligand 1 (PD-L1) inhibitor, set forth a multitude of clinical trials specifically in HPV-positive oropharyngeal SCC using immunotherapy as a first-line modality to “de-escalate” treatment from the standard chemotherapy and radiation. Unfortunately immunotherapy is only effective in 12-20% of head and neck cancers that are highly immunogenic, with an abundance of immune cells in the tumor microenvironment, while OSCC is poorly immunogenic and is therefore challenging to treat. For these reasons OSCC patients continue to have poor survival despite recent advances in head and neck cancer treatment. In certain embodiments, the REASON score is used as an adjunct measure to current clinical guidelines in determining the appropriate treatment for the patient. The REASON score cutoff is determined based on survival curves.

Mirroring the biomarker studies in breast cancer, head and neck cancer researchers have attempted to develop a multigene risk score to better tailor treatment for OSCC patients. Studies so far have used differential gene expression, gene amplification and deletions, methylation, and microRNA (miRNA) as potential biomarkers. In contrast to the embodiments herein, which identify high risk patients who would benefit from treatment escalation, the majority of studies have largely focused on preventing over-treatment by developing a biomarker to predict risk of neck metastasis. Currently the majority (up to 80%) of early stage OSCC patients do not have neck metastasis. However, 20% or more of these patients have occult (i.e., non-detectable by clinical exam or imaging) neck metastasis. Numerous publications, including computational modeling studies, retrospective studies, and one large prospective clinical trial that compares early stage OSCC patients who receive a prophylactic neck lymphadenectomy to those managed with a watch-and-wait approach, all demonstrate that the >20% risk of occult metastasis portends a poor survival in the absence of a prophylactic neck lymphadenectomy. As a result, it is current standard of care for early stage OSCC patients to receive a prophylactic neck lymphadenectomy, even if this practice involves over-treatment for up to 80% of patients with concomitant morbidity, including shoulder dysfunction, nerve damage and lymphedema. This clinical practice necessitates a need to develop a more nuanced approach of risk stratifying patients. However, to date no molecular signature exists that predicts risk of neck metastasis with high enough accuracy for use in a clinical setting. There is a need for biomarkers to predict poor survival in early stage OSCC

Rather than focusing on biomarkers to de-escalate neck dissections, methods disclosed here are directed to developing biomarkers of poor survival in early stage OSCC patients, with the intent of identifying high risk patients that might benefit from treatment escalation. The REASON score developed in this study predicts risk of death by 5 years in early stage OSCC patients with a c-index of 0.915. The risk score was developed by leveraging both a large internal cohort with publicly available TCGA data, focusing specifically on oral cavity sub-sites to maximize the likelihood of discovering meaningful biomarkers in a highly capricious disease. An internal cohort and a publicly available cohort were utilized to derive salient clinicopathologic factors with a 12-gene methylation signature to create the composite molecular/non-molecular REASON score, which has high prognostic performance in identifying early-stage (I/II) OSCC patients with high risk of death in 5 years.

While certain embodiments of the innovation have been shown and described herein, it will be obvious to those skilled in the art that such embodiments are provided by way of example only. The foregoing embodiments are illustrative examples. Numerous variations, changes, and substitutions will occur and be available to those skilled in the art without departing from the spirit and scope of the innovation. It should be understood that these various alternatives to the embodiments described herein may be employed in practicing one or more aspects of the innovation.

What is claimed is:

1. A method of providing decision support for a treatment regimen based on prognosis for an individual having oral squamous cell carcinoma (OSCC), the method comprising:
determining a high-Risk Epigenetic And clinicopathologic Score for Oral cancer (REASON) score from a biological sample from the individual having OSCC; and
selecting a treatment regimen in response to the REASON score, the treatment regimen being one or more of an elective neck dissection, radiation, immunotherapy, or chemotherapy.

2. The method of claim 1, wherein the individual has early-stage (I/II) OSCC.

3. The method of claim 1, wherein the REASON score is determined based on a plurality of non-molecular variables and a plurality of methylation patterns of a plurality of genes.

4. The method of claim 3, wherein the plurality of non-molecular variables includes one or more of age of the individual, sex of the individual, race of the individual, tobacco use by the individual, alcohol use by the individual, histologic grade of the OSCC, stage of the OSCC, perineural invasion, lymphovascular invasion, and margin status of the OSCC.

5. The method of claim 3, wherein the plurality of genes whose methylation patterns are determinative of the REASON score include two or more of ABCA2 (ATP-binding cassette sub-family A member 2), CACNA1H (Calcium Voltage-Gated Channel Subunit Alpha1 H), CCNJL (Cyclin-J-Like), GPR133 (Adhesion G-Protein-Coupled Receptor 133), HGFAC (hepatocyte growth factor activator), HORMAD2 (HORMA domain containing protein 2), MCPH1 (Microcephalin 1), MYLK (Myosin Light Chain Kinase), RNF216 (Ring finger protein 216), SOX8 (SRY-box transcription factor 8), TRPA1 (Transient Receptor Potential Cation Channel Subfamily A Member 1), and WDR86 (WD Repeat Domain 86).

6. The method of claim 1, wherein the biological sample is acquired using a brush swab.

7. The method of claim 1, wherein a poor prognosis is indicated for the individual with OSCC when the REASON score for the individual with OSCC is above a reference REASON score from a healthy individual.

8. The method of claim 7, wherein the REASON score ranges from zero to thirty-five.

9. The method of claim 8, wherein the reference REASON score is 17.

10. A method of risk stratification of an individual having oral squamous cell carcinoma (OSCC), the method comprising:
determining a high-Risk Epigenetic And clinicopathologic Score for Oral cancer (REASON) score from a biological sample from the individual; and
classifying the individual as having a high risk of OSCC-related mortality in response to the REASON score for the individual with OSCC being above a reference REASON score from a healthy individual.
